(19) United States
(12) Patent Application Publication
(10) Pub. No.: US 2013/0296739 A1
(43) Pub. Date: Nov. 7, 2013

(54) Diaper with Pocket for an Absorbent Pad Containing a Test Strip

(71) Applicant: Courtney Schultz, Shell Lake, WI (US)

(72) Inventor: Courtney Schultz, Shell Lake, WI (US)

(21) Appl. No.: 13/873,925

(22) Filed: Apr. 30, 2013

Related U.S. Application Data
(60) Provisional application No. 61/640,970, filed on May 1, 2012.

(51) Int. Cl. A61B 10/00; A61F 13/15

(52) U.S. Cl. CPC A61B 10/007; A61F 13/15
USPC 600/573

(57) ABSTRACT

Disclosed is a disposable absorbent product for use in testing for the presence of a medical condition, comprising a disposable diaper having a top flap formed of a liquid permeable layer and attached to the diaper on three sides, thereby forming a pocket. The pocket is designed to securely hold an absorbent pad containing a test strip in the crotch region of the diaper. The pocket enables the test strip to come into direct contact with urine or other bodily fluids released by a user in order to detect the presence of a medical condition. A variety of testing protocols can be used based on the expected medical issue, thereby providing a convenient and inexpensive means of detecting a medical condition. The device is particularly useful for patients who suffer from incontinence, mobility impairment, and dementia.

### CROSS REFERENCE TO RELATED APPLICATION

This application claims the benefit of U.S. Provisional Application No. 61/640,970 filed on May 1, 2012, entitled “Dependable Depends.” The above identified patent application is herein incorporated by reference in its entirety to provide continuity of disclosure.

### BACKGROUND OF THE INVENTION

1. Field of the Invention

The present invention relates to a diaper. More particularly, it pertains to an adult diaper with a pocket on the interior that allows for the insertion of an absorbent pad that can be used to test for the presence of a medical condition.

People who suffer from incontinence problems often use devices in order to prevent urine and feces from leaking through their underwear. One of the most commonly used products is an adult diaper, which is a diaper that is designed for use by a person with a body larger than an infant or toddler. Diapers are used by adults with various conditions, such as incontinence, mobility impairment, severe diarrhea or dementia. Adult diapers are made in various forms, including those resembling traditional child diapers, underpants, and pads resembling sanitary napkins, which are more commonly known as incontinence pads.

Adult diapers are generally utilized for medical conditions that cause a person to experience urinary or fecal incontinence, which is generally caused by an inability to control the bladder or bowels. Additionally, people who are bedridden or in wheelchairs, including people with normal bowel and bladder control, may also wear diapers due to an inability to access a toilet independently. A final group who utilizes adult diapers is those with cognitive impairment, such as dementia. Such a person may require diapers due to an inability to recognize the need to use a toilet.

In addition to incontinence, mobility impairment, and dementia, many people suffer from a variety of other medical issues, such as urinary tract or other types of infections, and sexually transmitted diseases. Some of these people, particularly those suffering from dementia, may not recognize that they have a medical problem, which can otherwise go untreated. This can weaken the immune system, and may lead to other health-related problems. While caregivers are trained to detect certain health-related problems in patients, they are not equipped to discover certain issues without input from a patient.

The present invention helps to overcome such problems with an adult diaper that provides a means of testing for a variety of medical conditions. The device comprises a conventional adult diaper with a pocket on the interior that allows for the insertion of an absorbent pad containing a test strip. The pocket enables the test strip to come into direct contact with urine or other bodily fluids released by a user in order to detect the presence of a medical condition. A variety of testing protocols can be used based on the expected medical issue, thereby providing a convenient and inexpensive means of detecting a medical condition.

2. Description of the Prior Art

Devices have been disclosed in the prior art that relate to adult diapers. These include devices that have been patented and published in patent application publications. These devices generally relate to adult diapers that prevent incontinence problems and can be used for detecting urinary tract infections. The following is a list of devices deemed most relevant to the present disclosure, which are herein described for the purposes of highlighting and differentiating the unique aspects of the present invention, and further highlighting the drawbacks existing in the prior art.

Specifically, Gael, U.S. Pat. No. 6,203,496 discloses a disposable diaper that has one or more chemical reagents provided in the absorbent region of the diaper. The reagents come into contact with urine when a patient wearing the diaper urinates. These chemical reagents change color when they contact urine containing abnormal levels of substances, such as leukocytes, blood, glucose, nitrites, protein, ketones, bilirubin, or urobilinogen; which may indicate the presence of a medical abnormality. The color change provides a visible indication of an abnormality in the urine and thus a possible incipient medical condition, such as a urinary tract infection, diabetes mellitus, hematuria, or the like. While the Gael device discloses a diaper with a color change indicator for detecting infections, it does not, however, include a pocket on the interior that allows for the insertion of an absorbent pad that can be used to test for the presence of a medical condition.

Similar to Gael, Everhart, U.S. Pat. No. 5,468,236 discloses a disposable absorbent product which includes a chemically reactive means having an end point adapted to provide a visual indication of the presence of a substance in mammalian bodily excrement, wherein the chemically reactive means is adapted to provide the visual indication as a result of interacting with the mammalian bodily excrement. The device can be constructed as a conventional, disposable, absorbent product, including tissues, wipes, and such wearable, disposable, absorbent products as diapers, feminine care products, incontinent care products, surgical gowns and drapes, and the like. The Everhart device utilizes a testing means that is built directly into the absorbent product.

Kritzman, U.S. Pat. No. 7,541,177 discloses a method for identifying physiological conditions associated with a biological fluid secreted from a person, by providing a secretion-monitoring article that includes an absorbent material for absorbing a biological fluid secreted from a person, and an indicator system having a hydrophobic chemical composition that includes an indicator agent and an ion-balance reagent of a quaternary amine. The user wears the article in a position to receive the biological fluid secreted from the person, which then detects a physiological condition associated with the biological fluid, as indicated by a measurement of the pH or buffer capacities of the fluid. This device detects the presence of an infection by measuring pH. The present invention can be used with a variety of test protocols for detecting a number of medical conditions.

In addition to the '177 patent, Kritzman, U.S. Pat. No. 7,314,752 discloses a secretion-monitoring article for identifying a secreted biological fluid. The article includes a body with an absorbent material for absorbing a biological fluid secreted from a person and an indicator system. The indicator system includes an indicator agent and an ion-balance reagent, wherein the indicator system provides an indication of physiological conditions associated with the pH or the buffer capacities of the biological fluid. The article can be embodied as a swab, gauze, shield, hygienic napkin, diaper or interlabial absorbent structure and can be used to indicate the presence of amniotic fluid, or secretions associated with bacterial vaginosis without giving a false positive result upon exposure to urine or after drying. This device, similar to the '177 device, detects the pH of a biological fluid for determining the presence of a medical condition, and does not disclose a diaper with a pocket on the inside.

The devices disclosed in the prior art provide adult diapers that can detect the presence of an infection. These devices utilize a variety of means of detecting infections. These prior art devices, however, include a means of testing for a particular infection or infections with a built-in test strip. They do not disclose a diaper with a pocket on the interior that allows for the insertion of an absorbent pad that can be used to test for the presence of a medical condition. The present invention enables medical personnel or caregivers to test for a variety of medical conditions by utilizing a pad containing a test strip that can detect the presence of various types of infections, or sexually transmitted diseases.

In light of the prior art and the disclosed elements of the present invention, it is submitted that the present invention substantially diverges in design elements from the prior art. Consequently, it is clear that that present invention is not described by the art and that a need exists for a disposable diaper that can be used to detect the presence of an infection. In this regard, the instant invention substantially fulfills these needs.

### SUMMARY OF THE INVENTION

In view of the foregoing disadvantages inherent in the known types of disposable diapers now present in the prior art, the present invention provides a new disposable diaper wherein the same can be utilized for providing convenience for the user when detecting the presence of a variety of medical conditions.

It is therefore an object of the present invention to provide a new and improved disposable diaper device that has all of the advantages of the prior art and none of the disadvantages.

It is another object of the present invention to provide a new and improved disposable diaper that can be used to detect the presence of a medical condition.

Another object of the present invention is to provide a new and improved disposable diaper that can be worn by users with incontinence problems, mobility impairment, and dementia.

Yet another object of the present invention is to provide a new and improved disposable diaper with a pocket on the interior that allows for the insertion of an absorbent pad that can be used to test for the presence of a medical condition.

Another object of the present invention is to provide a new and improved disposable diaper that may be readily fabricated from materials that permit relative economy and are commensurate with durability.

Other objects, features and advantages of the present invention will become apparent from the following detailed description taken in conjunction with the accompanying drawings.

### BRIEF DESCRIPTIONS OF THE DRAWINGS

Although the characteristic features of this invention will be particularly pointed out in the claims, the invention itself and manner in which it may be made and used may be better understood after a review of the following description, taken in connection with the accompanying drawings wherein like numeral annotations are provided throughout.

FIG. 1 is an overhead view of the present invention with the absorbent pad containing a test strip inserted into the diaper.

FIG. 2 is a close-up view of an absorbent pad placed within the pocket on the interior of the diaper.

FIG. 3 is a view of the present invention with the absorbent pad containing a test strip inserted into the diaper.

### DETAILED DESCRIPTION OF THE INVENTION

Reference is made herein to the attached drawings. Like reference numerals are used throughout the drawings to depict like or similar elements of the disposable diaper. For the purposes of presenting a brief and clear description of the present invention, the preferred embodiment will be discussed as used for detecting the presence of a medical condition. The figures are intended for representative purposes only and should not be considered to be limiting in any respect.

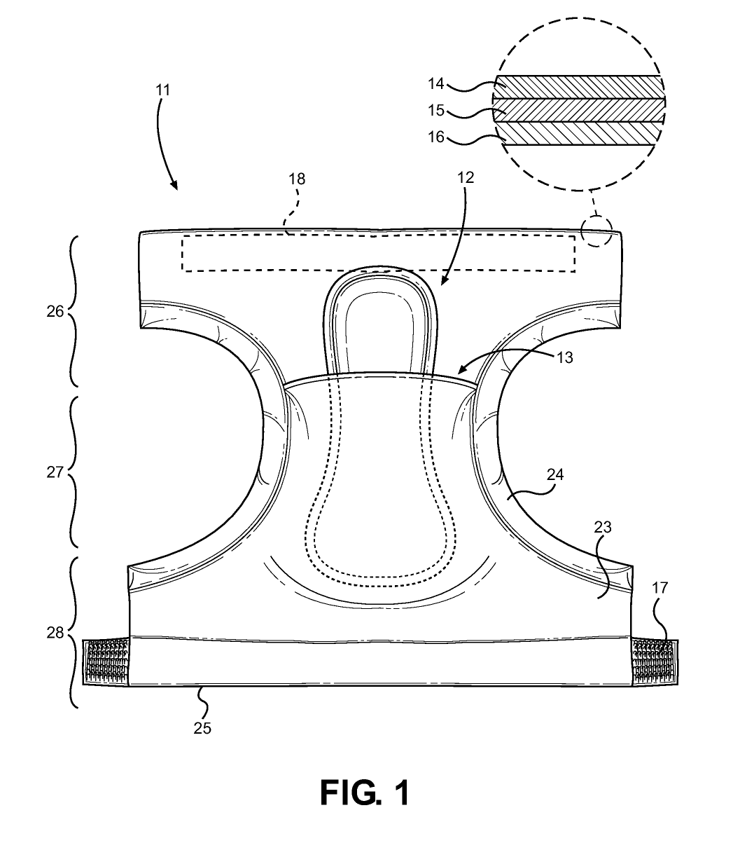

Referring now to FIG. 1, there is shown a view of the present invention, which is comprised of a disposable diaper 11, that can be used with an absorbent pad 12 containing a test strip. The pad is placed within a pocket 13 on the interior of the diaper, which securely holds the pad in position. The diaper preferably resembles a conventional diaper in appearance and function, or can optionally utilize the absorbent pad as the primary source of liquid absorption. When configured as a conventional diaper, the device includes a liquid permeable interior layer 14, an absorbent middle layer 15, and a liquid resistant outer shell 16. The device may be constructed with a fastening means that allows the diaper to be placed on a user without requiring the complete removal of the user's clothes, and may be fastened with hook 17 and loop 18 fasteners, adhesive tape, or another similar method. Alternately, the device can be constructed as a pull-up style diaper that is placed on a user in a similar manner to conventional undergarments.

The diaper is shown in a flat, relaxed state without elastic. The absorbent middle layer is situated between the liquid permeable interior layer and the liquid resistant outer shell. The diaper additionally includes side panels 23, elastic leg cuffs 24, and an elastic waist 25. The leg cuffs and waist each include an elastic member that provides improved fit and containment. The ends of the diaper are each generally configured as a first waist area 26, and a second waist area 28, while the area therebetween is generally configured as a crotch area 27 that extends longitudinally between the waist regions. The waist preferably includes elastic members that gather about the waist of the wearer to allow for the device to expand in order to fit on users of varying size. The crotch area is generally positioned between the user's legs, covering over the genital area and anus.

The liquid permeable interior layer is designed to allow urine and other bodily fluids that pass therethrough to be absorbed by the absorbent middle layer. The liquid permeable layer can be constructed from a variety of sheet-like materials having a liquid permeable structure, such as a nonwoven fabric, a woven fabric, perforated plastic sheet, or a mesh sheet. The liquid permeable layer makes direct contact with the skin of the user, and is designed to allow any liquid coming in contact therewith to pass through to the absorbent layer, which prevents rashes, irritations, and other skin problems from occurring due to prolonged contact with moisture.

The absorbent middle layer comprises an absorbent material that retains liquids such as urine, feces and other body fluids. The absorbent middle layer can be constructed from any variety of liquid-absorbent materials that are commonly found in disposable diapers, such as wood pulp, cellulose wadding, melt blown polymers, cellulosic fibers, tissue, absorbent foams, absorbent sponges, superabsorbent polymers, absorbent gelling materials or another similar absorbent material.

The liquid resistant outer shell is attached to the liquid permeable interior layer and absorbent middle layer. The outer shell acts as a barrier to prevent any bodily fluids released into the diaper by a user from leaking through the diaper and penetrating the exterior clothing or bed linens of the user. The outer shell is preferably constructed of a laminate of a nonwoven plastic film. Alternately, the device can be constructed from a breathable material that permits vapors to escape from the diaper while retaining bodily fluids therein.

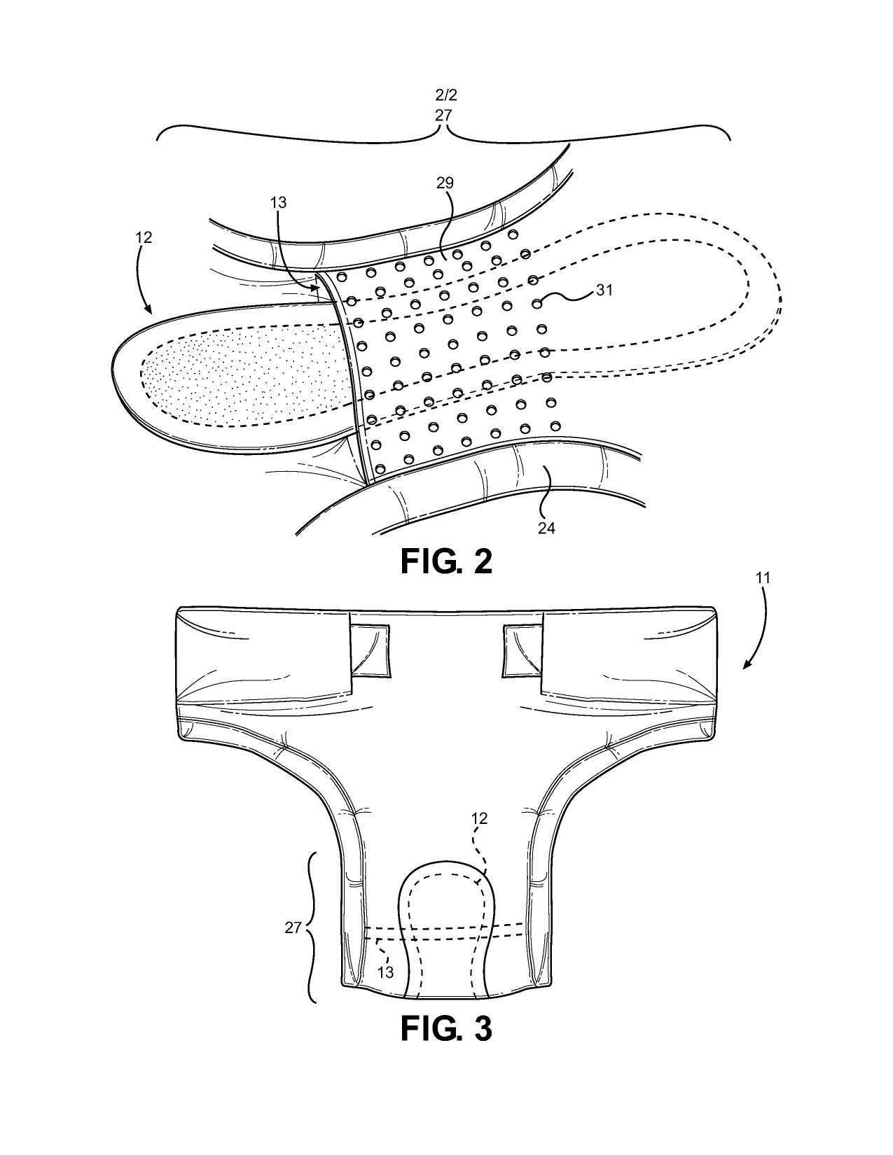

Referring now to FIG. 2, there is shown a close-up view of an absorbent pad placed within the pocket on the interior of the diaper. The crotch area 27 includes a pocket 13 for holding an absorbent pad 12 in place. The pocket is constructed of a top flap 29 that is attached to the diaper on three sides, with one free end. The top flap is preferably attached on either side to the elastic leg cuffs 24, and to the second waist area. The pocket is preferably constructed of a liquid permeable interior layer to allow urine and other bodily fluids to pass therethrough. This enables any fluids released by the user to pass through the top flap for absorption by the pad if positioned therein, or by the absorbent middle layer if no pad is present. The liquid permeable layer can be constructed from the same material as the liquid permeable interior layer.

The pocket provides a means of utilizing a variety of different absorbent pads containing a test strip for detection of various medical conditions. Because the test strip is distinct from the diaper, medical personnel or caretakers can select an appropriate testing protocol based on the suspected underlying condition. The appropriate absorbent pad can be inserted into the pocket, the device can be installed on the user, and the test strip can be check for the suspected medical condition at a later time. The pocket encompasses substantially the entire crotch area, thereby ensuring a universal fit for testing pads or strips of varying size. The top flap preferably includes a plurality of apertures 31 spaced about a substantial portion thereof, which are designed to allow urine and other bodily fluids to easily pass through the top flap and onto the pad. The apertures additionally enable the medical personnel or caretaker to view the pad in order to verify the presence of a medical condition, as would be indicated with a color changing pad.

Referring now to FIG. 3, there is shown a view of the present invention with the absorbent pad containing a built-in test strip inserted into the diaper. To use the present invention to test for a medical condition with an absorbent pad 12 that contains a test strip, a pad is placed within the pocket 13 on the interior of the diaper 11. The test strip should be selected based on the suspected underlying condition. Once the pad is inserted into the pocket, the diaper is installed on a user in a conventional manner. The pocket positions the pad so that it is positioned in the crotch area 27 to ensure appropriate contact with urine or other bodily fluids released or discharged by the user. The diaper and pad can be left in position until urine or other bodily fluids are released by the user. For cases of invalid patients who suffer from incontinence issues, the device should be left in position until the medical personnel or caretaker is confident that a sufficient amount of urine or bodily fluid has been released. If the user does not suffer from incontinence, the device can be left in place until the user indicates that he or she has released a sufficient amount of urine or bodily fluid. This is particularly useful for patients who do not suffer from incontinence, but are unable to use the restroom due to a medical condition.

Once the device has been in place for a sufficient amount of time, the diaper can be removed from the user and examined for the suspected condition. The medical personal can view the pad through the plurality of apertures in the top flap. Alternately, the top flap can be constructed of a thin enough material whereby the material becomes semi-transparent when wet, thereby providing a view of the pad contained therein. This prevents the medical personnel or caretaker from having to remove the urine soaked pad from the pocket for examination. Once the test is complete, the diaper can be discarded in a conventional manner.

Overall, the present invention provides a device that assists in the testing of various medical conditions. The device can be easily installed on a user, and can be utilized with a variety of test protocols. The device is particularly useful for patients who suffer from incontinence, mobility impairment, and dementia. When the proper test is installed in the pocket, the present invention can be used to test for a variety of medical issues, such as urinary tract or other types of infections, and sexually transmitted diseases. This enables medical personnel and caregivers to help patients that may not recognize that they have a medical problem, which would otherwise go untreated.

It is therefore submitted that the instant invention has been shown and described in what is considered to be the most practical and preferred embodiments. It is recognized, however, that departures may be made within the scope of the invention and that obvious modifications will occur to a person skilled in the art. With respect to the above description then, it is to be realized that the optimum dimensional relationships for the parts of the invention, to include variations in size, materials, shape, form, function and manner of operation, assembly and use, are deemed readily apparent and obvious to one skilled in the art, and all equivalent relationships to those illustrated in the drawings and described in the specification are intended to be encompassed by the present invention.

Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention.

I claim:

1. A disposable absorbent product for use in testing for the presence of a medical condition, comprising:
a disposable diaper having a first waist area, a second waist area, and a crotch area, said first waist area, second waist area, and crotch area each having a liquid permeable interior layer, an absorbent middle layer, and a liquid resistant outer shell; and
a top flap formed of a liquid permeable layer and attached to said diaper on three sides forming a pocket, said pocket being designed to securely hold an absorbent pad containing a test strip in said crotch region of said diaper.

2. The device of claim 1, wherein said diaper further comprises a fastening means.

3. The device of claim 1, wherein said diaper being constructed as a pull-up style diaper.

4. The device of claim 1, wherein said absorbent middle layer is placed between said liquid permeable interior layer and said liquid resistant outer shell.

5. The device of claim 1, wherein said diaper further comprising side panels, elastic leg cuffs, and an elastic waist.

6. The device of claim 1, wherein said top flap is attached on either side to said elastic leg cuffs, and to said second waist area.

7. The device of claim 6, wherein said top flap encompasses substantially the entire crotch area.

8. The device of claim 6, wherein said top flap further comprising a plurality of apertures spaced about a substantial portion thereof, said plurality of apertures allowing urine and other bodily fluids to pass through said top flap and onto said pad.

9. The device of claim 6, wherein said top flap further comprising a material that becomes semi-transparent when wet, thereby providing a view of said pad contained therein.
